(19) United States
(12) Patent Application Publication
(10) Pub. No.: US 2011/0218218 A1
(43) Pub. Date: Sep. 8, 2011

(54) Formulations Of Indanylamines And The Use Thereof As Local Anesthetics And As Medication For Chronic Pain

(75) Inventors: A.K. Gunnar Aberg, Sarasota, FL (US); Keith Johnson, Durham, NC (US)

(73) Assignee: BRIDGE PHARMA, INC., Sarasota, FL (US)

(21) Appl. No.: 13/081,100

(22) Filed: Apr. 6, 2011

Related U.S. Application Data
(63) Continuation-in-part of application No. 11/991,771, filed on Jun. 16, 2008.
(60) Provisional application No. 60/719,904, filed on Sep. 23, 2005.
(60) Provisional application No. 60/839,783, filed on Aug. 24, 2006.

(51) Int. Cl. A61K 31/4458; A61P 25/04

(52) U.S. Cl. 514/319

(57) ABSTRACT

Internal-dermal formulations of RS—, S—, or R-2-[2-(N-phenyl-N-2-indanyl)aminoethyl]piperidine causing intradermal accumulation of said piperidine and intended for the treatment of neuropathic pain are provided, as well as a method of inducing the relief of pain using the compounds.

This application is a continuation-in-part of U.S. Ser. No. 11/991,771 filed on Jun. 16, 2008, which claims priority of U.S. Provisional Application No. 60/719,904 filed Sep. 23, 2005 and U.S. Provisional Application No. 60/839,783 filed Aug. 24, 2006, the disclosures of which are incorporated herein by reference.

### BACKGROUND OF THE INVENTION

Local anesthetics inhibit batrachotoxin-sensitive neural sodium channels and are used to prevent expected, acute painful conditions. Compounds that inhibit specific voltage-sensitive sodium channels, such as NaV1.3 may be used to treat patients suffering from chronic pain, including neuropathic pain, provided said compounds can be delivered to the sites of action within the skin without causing dermal irritation or dermal toxicity.

The embodiments disclosed herein refer to new formulations containing the compound 2-[2-(N-Phenyl-N-2-indanyl)aminoethyl]piperidine (LAC-34) and the isomers thereof. LAC-34 is intended for use prevention and treatment of neuropathic pain. The formulations of the present invention transport LAC-34 from the surface of the skin and into the epidermis and the dermis layers of the skin, where the sites of action for LAC-34 are located. The present formulations are not transdermal formulations. The compound LAC-34 potently inhibits both batrachotoxin-sensitive sodium channels and the voltage-gated sodium channels NaV1.3, NaV1.5 and NaV1.8. The term “LAC-34”, as used herein, refers to the free base and the salt forms of racemic 2-[2-(N-Phenyl-N2-indanyl)aminoethylpiperidine and the optically active isomers thereof. Alternatively, the chemical name 2-{2-[N-(2-indanyl)-N-phenylamino]ethyl}piperidine can be used.

Local anesthetics are commonly administered by injection and are mainly used for the prevention of pain before surgery, but are also used for the treatment of pain (e.g. to inhibit or decrease intractable cancer pain.) Since LAC-34 is a potent inhibitor of NaV1.3, NaV1.5 and NaV1.8 (Example 20), this compound will be useful in preventing and treating neuropathic pain. Patients with neuropathic pain most often have intermittent pain and NaV1.3/1.5/1.8-inhibitors like LAC-34 have to be administered continuously and for several days to prevent the recurrence of pain in patients with intermittent types of neuropathic pain and to treat patients with persistent types of neuropathic pain conditions. Thus, compounds like LAC-34 have to be used both for prevention of expected pain and for treatment of existing pain. It is important that successful therapy of neuropathic pain with LAC-34 is not stopped prematurely, but the treatment has to be continued to prevent recurrence of the disease and the painful symptoms thereof. Approximately one out of five patients with shingles will develop postherpetic neuralgia (Postherpetic Neuralgia, Wikipedia 01-18-11, which publication is hereby included in its entirety by reference) and patients with shingles should be treated with NaV1.3-inhibitors, such as LAC-34 to prevent the development of post-herpetic neuropathies and the painful symptoms thereof. The mechanism of action of drugs and formulations thereof for the treatment of neuropathic pain, is not just to offer instantaneous inhibition of afferent nerve conduction, but the major purpose of the therapy is to inhibit the generation of pain signals in peripheral nerve endings and in the thin nerves that are located in the skin of patients suffering from neuropathies (neuropathic pain). Said thin afferent nerves and nerve-endings are distal branches of dorsal root ganglia (Dorsal root ganglion. (Wikipedia, 12-18-2010, which publication is hereby incorporated by reference.) The formulations of the present invention have been designed to make the transport of said chemical entities possible from the application site outside the skin to the biophase within the skin, where the active ingredient of the formulations (LAC-34) can exert therapeutic activities.

In combination with certain other compounds that can influence the function of sensory nerves, such as for example capsaicin, a useful potentiation of the therapeutic benefits has been found with regard to the effects on pain.

The terms “patch” and “dermal patch” are used as synonyms herein and refer to a device in the form of a medicated adhesive patch that is placed on the skin to deliver a medication, either across the skin (transdermal patch) or into the skin (intradermal patch.)

The term “topical anesthesia” is in this document defined as local anesthesia of mucosal membranes, such as for examples those of the eye, the ear, the mouth, the nose, the rectal area and the urogenital tract. The term “dermal anesthesia” is in this document refers to treatment of nociceptive or neuropathic pain in the skin. The term “infiltration anesthesia” refers to anesthesia of thin nerve fibers and nerve endings by infiltrating the tissues containing such nerves or nerve endings with a formulation containing a local anesthetic compound. The term “nerve block” in this document refers to the total or partial blockade of nerve transmission by administering a formulation of local anesthetic compound close to said nerve. The term “local anesthesia” in this document includes all anesthesia of afferent and efferent nerves and includes “dermal anesthesia”, “topical anesthesia” “infiltration anesthesia” and “nerve blocks”.

The terms “neuropathic pain” and “neuropathy” refer to a group of chronic painful conditions characterized by pain originating from neural damage. The debilitating symptoms of thermal and/or mechanical hyperalgesia (allodynia) are typical for neuropathic diseases.

The various conditions of neuropathic pain have presented clinicians with multiple challenges ranging from difficulties in diagnosis to a lack of effective treatments. When the diagnosis has been made, clinicians typically resort to treating neuropathic pain with old medications that were originally developed for other indications. Due to the lack of effective medications, treatment decisions are often made on a trial and error basis and therapeutic agents, such as tricyclic antidepressants, gabapentin, lidocaine patches, narcotics such as oxycontin, and painkillers such as tramadol are being used. The development of improved experimental models and a better understanding of the pathophysiology of the different neuropathic pain conditions has recently made the rational testing possible of compounds that may be useful for neuropathic pain. Additionally, clinical evaluation of these and existing agents in well-defined patients groups will allow treatment guidelines to further improve. Better knowledge of various types of neuropathic pain will allow the scientific community to select target indications of drugs for neuropathic pain, such as for example neuropathic low back pain (Barker 2011, which publication is hereby included by reference), diabetic neuropathy (Wikipedia 02-13-11, Diabetic neuropathy, which publication is hereby included by reference), HIV—related neuropathic pain (Nam aidsmap: Neuropathy—nerve pain, April 2010, which publication is hereby included by reference), postherpetic neuralgia (Wikipedia 01-18-11, Postherpetic Neuralgia, which publication is hereby included by reference) and various other neuropathic conditions (About.com, Common types of neuropathic pain. 2011, which publication is hereby included by reference.)

Gabapentin and similar compounds are being used in patients suffering from Neuropathic Low Back Pain (NLBP), while lidocaine patches may offer relief of Post Herpetic Neuralgias (PHN). However, and as mentioned above, NSAIDs, narcotics, and even antidepressants are presently used by patients and doctors who desperately try to find medication to decrease the intensity of neuropathic pain. Parenteral injections or dermal application of formulations containing capsaicin or similar agents that may have effect on interneurons in the substantia gelatinosa of the dorsal spinal cord offer promise.

The drug described herein, LAC-34, is useful both as a local anesthetic and as medication for patients suffering from neuropathic pain. When used as a local anesthetic (and particularly as a dermal anesthetic), it is important that the formulation of LAC-34 offers a short onset time of anesthesia. On the other hand, when used as medication for chronic neuropathic pain, the emphasis is on long duration of activity.

The preparation of effective, efficient formulations containing LAC-34 has proved to be challenging due to the unusual solubility profile of this active moiety. Thus, the free base of LAC-34 has too low water solubility for injection, but too high solubility for use in intradermal formulations. Pharmaceutically acceptable salts may exist that have improved water solubility. However, in preclinical studies it has now been found that the free base of LAC-34 offers advantageous duration of action when compared with various salt forms of said compound (Example 21). The salt forms described in U.S. Pat. No. 7,718,674, are useful for LAC-34 and the isomers thereof. Said U.S. Pat. No. 7,718,674 is hereby incorporated by reference.

In accordance with embodiments disclosed herein, dermal compositions have now been formulated, such as solutions, creams, gels (ointments) and aerosols containing LAC-34. The free base and practically all known salt forms of LAC-34 are insoluble in water. An exception is the dihydrochloride salt that is water-soluble and can be used in aqueous solutions for injections. The new formulations express (a) inhibition of batrachotoxin-sensitive neural sodium channels (local anesthetic activities), making them useful as dermal and topical anesthetics and (b) inhibition of NaV1.3, NaV1.5 and NaV1.8 neural sodium channels, making them useful for the treatment of chronic pain, in particular for the treatment of neuropathic pain. The intradermal formulations typically contain LAC-34 in combination with one or more solvents, one or more anti-solvents, carriers, penetration enhancers, occlusive agents, and/or emollients. The combination of excipients provides means of delivering the drug from highly concentrated (e.g., supersaturated) compositions through the penetration barrier (stratum corneum) and into the epidermis and dermis layers of the skin, where the sites of therapeutic activity are located for LAC-34, when used for neuropathic pain. The free base of LAC-34 is preferred, but pharmaceutically acceptable salt forms may also be used (Example 21).

The combination of oral or parenteral medications for neuropathic pain with one or more drugs that are encompassed by the embodiments disclosed herein may result in improved therapeutic activity. For example, LAC-34 by itself or in combination with capsaicin, when administered intra-dermally or parenterally can be combined with a drug such as for example gabapentin (Neurontin®, Pfizer), phenyloin (Dilantin®, Pfizer) or carbamezepine (Tegretol®, Novartis) that is dosed orally to the patient.

### SUMMARY OF THE INVENTION

The embodiments disclosed herein relate to intra-dermal formulations containing LAC-34, to methods of using said formulations as local anesthetics, methods of inducing local, topical or dermal anesthesia, methods of administering the formulations, particularly to a localized region of a patient, and for the treatment of pain and in particular, chronic pain.

The chemical structure of LAC-34 is:

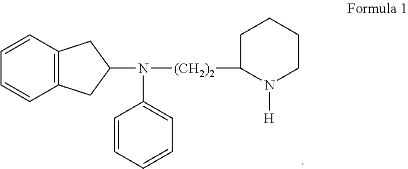

### LAC-34

The chemical compound LAC-34 (2-[2-(N-Phenyl-N-2-indanyl)aminoethyl]piperidine) exists as a free base as well as numerous salts. LAC-34 is an amine and is not metabolized in dermal tissues. The compound has pharmacological properties that render it useful as a potent and longacting inhibitor of batrachotoxin channels (inhibition of nociceptive pain) and of the sodium channels NaV1.3, NaV1.5 and NaV1.8 (inhibition of neuropathic pain). LAC-34 achieves short onset time and long duration of local anesthesia, topical anesthesia and dermal anesthesia.

It has now been found that the solubility profile of LAC-34, as the free base, in many solvents, can be effectively reduced by the addition of an anti-solvent, in an amount sufficient to entice the LAC-34 active agent, upon dermal or topical application, to readily penetrate the skin of a patient and reach the nerve structure within the skin in a sufficient concentration to achieve a therapeutic effect. It was found that water is potently decreasing the very high solubility of LAC-34 in numerous solvents, thereby making the formulations near-saturated with regard to LAC-34. Importantly, the free base LAC-34 and most salts thereof are not water-soluble and the single purpose of adding an anti-solvent is to reduce the solubility of LAC-34 in the solvents of the formulation and thereby increase the saturation of LAC-34 in said formulations. Thus, even small amounts of the selected anti-solvent reduce the solubility of the free base of LAC-34 dramatically, such that LAC-34 in the formulation approaches saturation or becomes saturated, enhancing the propensity of the active agent to come out of solution and enter into the skin. Indeed, the solubility of the free base in various solvents is so high that the free base is virtually ineffective as a dermal or topical anesthetic agent when dissolved in such solvents in the absence of an anti-solvent for LAC-34. The anti-solvent therefore increases the rate at which the LAC-34 penetrates the skin of a patient compared to identical formulations that are devoid of the anti-solvent.

Formulations for parenteral injections of solutions containing LAC-34 or an isomer thereof, contain said compound preferably as a water-soluble salt in a solution that may also contain a preservative, a buffer and/or a vasoconstrictor.

In combination with other compounds, such as capsaicin, a surprising improvement of the therapeutic activity has been found.

Prevention and treatment of (a) acute pain, such as for example pain caused by circumcision, vaccination, venopuncture, intravenous cannulation and (b) chronic pain, such as for example neuropathic pain, in particular dermal neuropathic pain, using the formulations disclosed herein may be achieved by repeatedly applying topical formulations containing LAC-34 on the skin or by injecting solutions of LAC-34 to infiltrate biological tissues in the vicinity of nerves.

Neuropathic pain can be intermittent or constant, although many patients have constant pain with additional recurrent sharp, often knife-like burst of intermittent pain. It is evident that prevention of expected pain is the most common use of local anesthetics, such as LAC-34, when used for acute pain—this is true regardless if it is an infiltration anesthesia or a nerve block, such as before the dentist goes to work on the teeth of a patient, or if dermal anesthesia is induced before injections, vaccination or circumcision that are expected to cause pain.

Prevention of neuropathic pain is a purpose of intradermal administration of LAC-34 to patients, because neuropathic pain is an intermittent/recurrent disease with periods of intense pain, followed by periods with less or no pain (e.g., “good days”). In order for the tissue concentrations of LAC-34 to accumulate and exceed the pain thresholds, it is important for the patient that the medication is taken continuously, even preventively during periods with less or no pain, e.g., “good days”. The preventive use of NaV1.3/1.5/1.8 sodium channels inhibitors, like LAC-34 and lidocaine, have to be continued for several days or weeks before the beneficial effects on neuropathic pain is optimal (Lidocaine Packet Insert, Endo, 2006, which document is hereby included by reference.) The progress of the preventive treatment sometimes can be difficult to establish because of the intermittent pain and pain-free periods. It is, however of pivotal importance that the preventive medication is not stopped or interrupted, which will delay the onset of the beneficial effects on neuropathic pain of any type. As an additional example, preventive drug administration should be initiated in diabetic patients with early signs of PDN (painful diabetic neuropathy) in order to prevent the more severe types of the disease that will develop over time. The drug will also be used by patients suffering from shingles, for the dual purpose of offering acute relief from the pain of shingles and the prevention of the appearance of postherpetic neuralgias that are usually a late complication of shingles, appearing days or weeks after the shingle pain has subsided.

The compound LAC-34 can also be used as oral therapy for patients suffering from pain, such as for example neuropathic pain. Conventional oral dose forms may be used and controlled release oral formulations may have advantages over regular tablets or capsules. The oral dose of LAC-34 will have to be titrated for the weight and age of the patient, the severity of the condition and the results that may be expected. Oral doses of between 3 mg and 300 mg may be useful, but lower doses may also be used.

Formulations of LAC-34, intended for dermal and topical administration to patients with neuropathic pain, preferably contain LAC-34 in its free base form of reasons shown in EXAMPLE 20, herein. However, several pharmaceutically acceptable salt forms, such as for example the mesylate or the monohydrochloride and other salts with limited water solubility but suitable solubility in various other excipients, may be used for dermal and mucosal administration of LAC-34. The optical isomers of LAC-34 have certain advantages over the racemic compound LAC-34 as disclosed in U.S. Pat. No. 6,413,987. Statements and test results regarding formulations for racemic LAC-34 in this document are valid also for the optically active isomers of LAC-34. Thus instead of the racemic mixture LAC-34, the optically active isomers thereof can be used in the formulations described herein.

It was found that the acute and severe pain caused by parenteral or dermal administration of capsaicin is eliminated by administration of a composition, containing LAC-34 as the single therapeutic agent or a formulation containing both LAC-34 and capsaicin. Embodiments disclosed here in present that capsaicin can be conveniently administered together with LAC-34 in the same formulation.

Certain embodiments relate to a method of inducing relief of neuropathic pain in a patient in need thereof, comprising applying to the skin of said patient a therapeutically effective amount of 2-[2-(N-phenyl-N-2-indanyl)aminoethyl]piperidine, or an optically active isomer thereof or pharmaceutically acceptable salt thereof, dissolved in one or more solvents in which the piperidine (or isomer or salt) is soluble, together with an anti-solvent in an amount sufficient to reduce the solubility of the 2-[2-(N-phenyl-N2-indanyl)aminoethyl]piperidine (or isomer or salt) in the one or more solvents. Application of the formulation to the skin of a patient causes the solvent to evaporate to form a saturated solution of the 2-[2-(N-Phenyl-N2-indanyl)aminoethyl]piperidine (or isomer of salt), causing the 2-[2-(N-phenyl-N-2-indanyl)aminoethyl]piperidine (or isomer or salt) to penetrate the stratum corneum and to accumulate in the epidermis and dermis of the skin (the biophase) in an amount effective for inducing relief of neuropathic pain.

Certain embodiments relate to a method of down-regulating the up-regulated sodium channels in thin dermal afferent nerve fibers and in dermal nerve endings of the skin of a patient suffering from neuropathic pain. Such embodiments include applying to the skin of the patient a therapeutically effective amount of 2-[2-(N-phenyl-N2-indanyl)aminoethyl]piperidine, or an optically active isomer or pharmaceutically acceptable salt thereof, dissolved in one or more solvents in which the piperidine (or isomer or salt) is soluble, together with an anti-solvent in an amount sufficient to reduce the solubility of the 2-[2-(N-phenyl-N2-indanyl)aminoethyl]piperidine (or isomer or salt) in the one or more solvents at least one of which is a volatile solvent. Application of the formulation to the skin of a patient causes the volatile solvent to evaporate to form a saturated solution of the 2-[2-(N-Phenyl-N2-indanyl)aminoethyl]piperidine (or isomer of salt), causing the 2-[2-(N-phenyl-N-2-indanyl)aminoethyl]piperidine (or isomer or salt) to enter into the skin, penetrate across the stratum corneum and accumulate in the epidermis and dermis of the skin.

### DETAILED DESCRIPTION OF THE INVENTION

### 1. Neuropathic Pain

Examples of neuropathic pain diseases (neuropathies) are post-herpetic neuralgias, painful diabetic neuropathies, phantom pain syndromes, HIV-associated neuropathies and fibromyalgia rheumatica. Other forms of neuropathies exist.

The indanylamine compounds of the instant application are intended for dermal application to mammals, mainly humans, suffering from nociceptive pain or neuropathic pain (neuropathies). Neuropathic pain is vastly different from normal pain (called “nociceptive pain”). Nociceptive pain is caused by painful stimuli, such as for example acute trauma and is a normal reaction of the body, making the brain aware of a possibly dangerous situation. The nociceptive pain signals are sensed by afferent (sensory) nerve endings and the pain signals are forwarded via afferent nerves to the brain, where the signals are amplified and processed. Neuropathic pain, on the other hand, is a serious disease and is completely different from the normal, nociceptive transmission of warning signals to the brain. Thus, the severe pain signal transmitted to the brain in patients with neuropathic pain are actually generated by the nerves themselves, which can happen in nerves that have been damaged, for example by a virus infection or by trauma. It is important to realize that the two types of “pain” are completely different and are also treated with different types of medication as patients suffering from neuropathic pain are not at all helped by NSAIDS, such as aspirin, ibuprofen, acetaminophen or other over-the-counter pain medications. Nociceptive pain is sensed through activation of normally functioning neural sodium channels that are often referred to as batrachotoxin-sensitive sodium channels. Neuropathic pain is caused by malfunctioning of specific neural voltage-sensitive sodium channels, in particular NaV1.3, NaV1.5 and NaV1.8. It has been pointed out that both tetrodotoxin-sensitive and tetrodotoxin-resistant sodium channels may be involved in various types of neuropathies (Hong et al., 2004, which publication is hereby included by reference), and it is commonly agreed that said sodium channels (NaV1.3, NaV1.5 and NaV1.8) are of major importance and are up-regulated (meaning increased in numbers and activity) in patients suffering from neuropathic pain. Anatomically, upregulated sodium channels can be located in thin afferent nerve fibers and in sensory nerve endings in the epidermis and the dermis layers of the skin. The sodium channel upregulation usually follows a virus infection (ex. postherpetic neuropathies, HIV neuropathies) or trauma (ex. phantom pain, postsurgical neuropathic pain). The up-regulated sodium channels are initiating the neuropathic pain signals, which are transmitted to the brain. The compounds of the present invention have therapeutic activity by causing inhibition and down-regulation of the up-regulated sodium channels in thin dermal afferent nerve fibers and in dermal afferent nerve endings.

2. Intradermal vs. Transdermal Drug Transport.

The current formulations will cause an intradermal (actually intra-epidermal) accumulation of LAC-34. This is in stark contrast to transdermal formulations of various drugs, which merely is a transport across the skin of drugs that are absorbed by capillary blood vessels in subcutis and transported by the blood to the various biophases of such drugs. It should be noted that there are no direct transport systems nor are there any possibilities for direct diffusion of any drugs from the subcutis to various biophases in the body, such as for example a knee or the brain. Transdermal formulations are usually fairly simple and usually consist of a solvent containing a drug that can cross the various layers of the skin, whereupon the mixture of the solvent and the drug are absorbed by the capillaries in subcutis and distributed systemically in the body. This is vastly different from the instant formulations that are designed to force the drug (LAC-34) out of a hypertonic (supersaturated) dermal formulation on the surface of the skin and into those layers of the skin (epidermis and dermis) where the compound exerts its therapeutic activity.

Transdermal formulations of drugs may constitute “oil-continuous solution” with the drug dissolved in both the water- and oil-phases of the formulations. This requires an acceptable water solubility of the drug. As an example, Toppo (U.S. Pat. No. 5,985,860) presented a series of transdermal formulations for antihistamines and nonsteroidal anti-inflammatory drugs (NSAIDs). The minimum requirement for water solubility of drugs to be included in Toppo's formulations was stated as 1 mg/ml. The water solubility of LAC-34 is 0.005 mg/ml, as shown in Table 3. Thus, formulations of the type described by Toppo in U.S. Pat. No. 5,985,860 cannot be used for compounds like LAC-34. The Toppo formulations of various antihistamines and NSAIDs were transdermal while the instant formulations of LAC-34 are not transdermal but offer intradermal accumulation of the compound LAC-34.

### 3. Transport of LAC-34 to the Biophase

The dermal nerve structures causing neuropathic pain are mainly located in the epidermis, although thin afferent nerve fibers are also be residing in the dermis.

Importantly, the epidermis is the biophase (meaning: site of therapeutic activity) for LAC-34 when applied dermally for neuropathic pain and the purpose of dermal formulations described herein is to cause an accumulation of LAC-34 in the epidermis, without causing dermal tissue toxicity, which might further aggravate the neuropathic pain for the patient.

### 4. Stratum Corneum, Epidermis and Dermis

The epidermis is the outermost part of the skin and consists of five anatomically different layers of which the top layer is the epidermal barrier, often called the stratum corneum. This layer is the dermal penetration barrier that in part consists of dead cells and in part of living dermal cells (corneocytes) that are locked together by lipid lamellae and specialized cells (corneodesmosomes) that provide tensile strength and resist shearing forces. Elias (1983, which publication is hereby included by reference) described stratum corneum as being similar to a brick wall, with the corneocytes were the bricks, the lipid lamellae acted like mortar and the corneodesmosomes were analogous to iron rods that pass down through holes in the bricks, offering tensile strength to the stratum corneum. The remaining four layers of the epidermis consist of a multitude of living cells and are most often referred to as “epidermis” and it is in these epidermal tissues that thin afferent nerves and nerve endings are residing. The dermis is located below the epidermis and thin afferent nerves are also residing in this layer of the skin. The term “cutis” refers to the epidermis+dermis and the term “subcutis” refers to the innermost dermal layer of the skin, which is vascularized with capillary blood vessels.

### 5. Formulations of LAC-34

As mentioned above, the purpose of the present formulations is to cause an accumulation of LAC-34 molecules in the epidermis, where said molecules will prevent an up-regulation or cause a down-regulation of neural sodium channels that are up-regulated in patients suffering from neuropathic pain. Thus, the dermal formulations of the instant claims have been developed which allow for (A) transfer of the active ingredient through the penetration barrier (stratum corneum), followed by (B) intradermal accumulation of the active ingredient in the epidermis and dermis, which tissues contain the thin nerve fibers and the nerve endings that are causing neurophatic pain. Thus, the instant formulations are made to cause accumulation of the indanylamines within the skin.

The dermal and topical formulations of the embodiments disclosed herein contain the compound LAC-34 or an optically active isomer thereof in concentrations effective for inducing local anesthesia, and particularly in concentrations of about 0.05% to about 30%, preferably 0.1% to 10%. Dermal formulations containing LAC-34 may be applied on the skin one to four times daily, depending on the severity of the disease, the condition of the patient and/or the effect that is sought. Those skilled in the art will realize that dermal formulations containing 5 percent or more of RS—, S— or R-LAC-34 may be used less often than once daily and in some patients only once or twice weekly.

Dermal formulations containing a capsaicinoid such as capsaicin contain said capsaicinoid in concentration between about 0.001% and about 5%, preferably 0.01% to 1.0%. As realized by those skilled in the art, higher or lower concentrations of both LAC-34 and a capsaicinoid may prove to be useful and, under certain circumstances, even preferred. Dermal formulations containing LAC-34 in combination with a selected capsaicinoid, such as for example capsaicin, may be applied on the skin one to four times daily, depending on the severity of the disease the condition of the patient and the effect that is sought. The concentrations of capsaicin may be increased since the presence of LAC-34 will mask local side effects of capsaicin.

Solutions for dermal application contain one or more excipients in addition to LAC-34 or an optically active isomer of LAC-34. A partial list of suitable pharmaceutically acceptable excipients is shown in Table 1.

TABLE 1
Examples of excipients, evaluated for their usefulness in formulations
of LAC-34 (including solutions, gels and creams/lotions).
The term “volatile solvent” refers to a nonaqueous liquid with
solvents with the distinctive characteristic of evaporating readily
at normal skin temperature (32-34° C.)

| Excipient | Description | Function |
|---|---|---|
| Capric/caprylic | Semi-polar oil | Oil phase for emulsion, |
| triglycerides | | emollient |
| Dibutyl adipate | Semi-polar oil | Solvent, emollient |
| DMSO | Solvent | Solvent, penetration enhancer |
| Ethanol | Volatile solvent | Solvent, penetration enhancer |
| Hexylene glycol | Solvent | Solvent |
| Isopropyl | Semi-polar oil | Oil phase for emulsion, |
| myristate | | emollient |
| Isopropanol | Volatile solvent | Solvent, penetration enhancer |
| Mineral oil | Oil | Oil phase for emulsion, semi- |
| | | occlusive |
| Pentane | Oil | None, used as model propellant |
| Propylene | Solvent | Solvent |
| carbonate | | |
| Propylene | Solvent | Solvent, penetration enhancer, |
| glycol | | humectant |
| Triacetin | Semi-polar oil | Solvent |
| Water | Water | Anti-solvent |

As known by those skilled in the art, practically all chemical entities have limited solubility in one or more of the key formulation excipients. Surprisingly, this was not the case for LAC-34. This compound was soluble in all the excipients screened, with the exception of mineral oil, as shown in Table 2 (EXAMPLE 2). In addition, the free base of LAC-34 proved to be practically insoluble in water (Table 3.) This made it highly unlikely to find useful prior art formulation for LAC-34.

The addition of an anti-solvent to non-aqueous LAC-34 formulations was found to efficiently and effectively lower the solubility of the LAC-34 which will improve the ability of the drug to penetrate from the surface of the human skin into the skin. Water was found to be an excellent anti-solvent for LAC-when dissolved is selected excipients. The decreased solubility in the presence of water is not merely due to the decreased concentration of the solvents, since a very significant decrease of the solubility of LAC-34 was obtained when only small amounts of water are added. Thus, water did not merely dilute the solvents, but water acted as an anti-solvent for LAC-34 in the investigated excipients.

Solvents and penetration enhancers are useful in various formulations when combined with LAC-34, including: capric/caprylic triglycerides, dibutyl adipate, DMSO, ethanol, hexylene glycol, isopropyl myristate, isopropanol, mineral oil, pentane, propylene carbonate, propylene glycol, triacetin, water, decylmethylsulfoxide, N,N-dimethyl acetamide, 2-pyrrolidone, N,N-dimethyl formamide, 1-methyl-2-pyrrolidone, 5-methyl-2-pyrrolidone, 1,5-methyl-2-pyrrolidone, 1-ethyl-2-pyrrolidone, 2-pyrrolidone-5-carboxylic acid, oleic acid, laurocapram (azone), limonene, cineole, diethyl-m-toluamide (deet), sodium dodecylsulfate, trimethyl phosphine oxide, tetrahydrofurfuryl alcohol, glycerol, monolaurate, methyl oleate, and propylene glycol monolaurate. The preferred concentration of solvents in dermal formulation will range from 0.4 percent to 99.6 percent, and will depend on which excipients are being used.

Many penetration enhancers are also surfactants and based on the current findings of physicochemical properties of LAC-34, the following selected anionic surfactants pair with LAC-34 in various formulations: sodium dodecylsulfate, dioctyl sodium sulfosuccinate, triethanolamine lauryl sulfate and ammonium lauryl sulfate. The preferred concentration of surfactants in dermal formulation of LAC-34 will range from 0.4 percent to 99.6 percent, and will depend on which additional excipients are being used and the concentration of said additional excipients.

Those skilled in the art will appreciate that certain non-ionic surfactants may be useful in various dermal formulations. Examples of such surfactants are: glycerol monolaurate, propylene glycol monolaurate, sorbitan monolaurate, sorbitan sesquioleate, polysorbate 20, peg-40 stearate, steareth-20, poloxamer 185, sodium lauryl sulphate. In addition, selected cationic surfactants, such as for example cetyl pyridinium chloride and benzalkonium chloride will also pair with LAC-34. Zwitterionic surfactants, such as for example lecithin will also be useful.

Various “solubilizers” may be used and are generally used to improve the solubility of a drug in the selected vehicle and/or improve the dermal penetration and/or act as humectants. Examples of “solubilizers” are neutral methacrylic acid esters, polyhydric alcohols such as propylene glycol or polyethylene glycol, surfactants such as sodium lauryl sulphate, vitamin E or combinations of various solubilizers.

Permeation enhancers will improve the dermal penetration and are usually lipophilic solvents, such as for example dimethylsulfoxide, or a surfactant such as for example sodium lauryl sulfate or tween, oleic acid, oleic acid/PG, octyl dimethyl para-amino benzoic acid, a polyhydric alcohol such as propylene glycol or combinations of various penetration enhancers.

Plasticizers may be added to improve the softness of the dermal film and may consist of for example PG, polyethylene glycol, dimethylisosorbide acetyltributyl citrate, triethyl citrate or combinations of plasticizers.

Humectants can be added to promote the retention of moisture in the skin and examples of humectants are polyhydric alcohols, such as for example butylene glycol, propylene glycol, polyethylene glycol, sorbitol and glycerol, or combinations of various humectants.

Anti-solvents can be added to decrease the solubility of molecules in various solvents and examples of anti-solvents are for example ethylene glycol, diethylene glycol, glycerol, 1.2-propandiol, isopropanol and water. In the case of LAC-34, water was found to be an excellent anti-solvent as shown in Table 4. The concentrations of anti-solvents needed in the present formulations will depend on the concentration(s) of the solvent or solvents in the formulation and the efficacy of the selected anti-solvent in said solvent(s). Depending on the specific circumstances, the concentrations of the anti-solvent may range from 0.5 percent of the concentration of the solvent(s) up to 90 percent of the concentration of a specific solvent when other solvents are present. Most often it is enough if the anti-solvent constitutes from 1 percent to 10 percent of the final formulation, but higher (up to 50 percent) or lower concentrations (as low as 0.1 percent) of the anti-solvent may be used as obvious to those skilled in the art of making dermal and topical formulations. Under some conditions (such as for example a formulation intended for use as a topical anesthetic) no anti-solvent may be needed.

Suitable polymers may be included in the formulation to form a stable film on a surface, such as skin, when applied. Preferred film-forming polymers include methacrylic polymers and copolymers as well as acrylic polymers and copolymers, such as for example a copolymer of dimethylamine ethyl methacrylate and a neutral methacrylic acid ester, ammonio methacrylate copolymer type A or type B, methacrylic acid copolymer type A or type B, hydroxypropylcellulose, hydroxyethylcellulose, methyl or ethyl cellulose, cellulose acetate, polyvinyl alcohol and povidone. The porosity of the film is considered important and can be increased by adding water-soluble compounds, such as for example propylene glycol, polyethylene glycol, sodium lauryl sulfate, cetomacrogol or transcutol, or combinations thereof.

Where the formulation is an aerosol, the propellant used may be any pharmaceutically acceptable propellant. Preferred propellants include for example butane, isobutene, propane, dimethylether or other hydrocarbons, dichlorodifluoromethane, trichloro-monofluoromethane, dichloro-fluoroethane, difluoroethane, monochloro-difluoro-methane, dichloro-tetrafluoroethane, hepta-fluoropropane, tetrafluoroethane or other fluorocarbones, or a compressed gas, such as for example carbon dioxide or nitrogen.

Aerosol formulations of the present invention cause low skin irritation and can be used on hypersensitive skin, such as skin with shingles/allodynia. Aerosol formulations and aerosol dispensers are easy of use and relatively inexpensive to manufacture.

The aerosol dispenser can be a conventional aerosol can with a conventional metered spray aerosol valve. The pump dispenser can be a conventional bottle or can with a conventional metered spray pump.

An aerosol composition is typically applied over a fixed, predetermined area of the skin and is usually from 10 cm² to 25 cm² per actuation. Multiple actuations will be used to cover larger areas, such as for example to patients suffering from shingles or forms of neuropathic pain.

An aerosol or spray device can also be used to deliver a foam formulation of LAC-34. Dermal and/or topical foam formulations can also be developed and delivered by use of devices that are identical or similar to those described by R. C. Davis in U.S. Pat. No. 5,143,717, which is hereby included by reference. Numerous other aerosol and spray devices exist and can be used, as is well known to those skilled in the art of dermal and topical medicinal delivery.

A dermal or topical formulation of LAC-34 may include DMSO or a similar penetration enhancer. Animal experience have indicated that the vehicle may contain as much as 97% DMSO, although most biological studies were performed with vehicle containing 95% DMSO+5% water or less DMSO.

### Suspension Formulations

LAC-34 and its salts, or optically active isomers thereof, could also be prepared as suspensions in dermal formulations. In these formulations, LAC-34 would be present as fine to coarse particles with suitable stabilizers and other excipients. The term “nanosuspensions” refers to colloidal dispersions of sub-micron-size particles of a drug, which are most often stabilized by one or more surfactants in the suspension. Sub-micron size particles of a drug such as LAC-34 may also be dispersed in lipidic carriers. Nanosuspensions are of interest for LAC-34 since such suspension may be supersaturated with LAC-34. Such a supersaturated suspension may be stable until intentionally destabilized to form saturated or supersaturated dermal formulation. Suspension formulations with particles of LAC-34, its salts or optically active isomers, wherein said particle sizes may range from coarse to submicron size, are encompassed in the embodiments disclosed herein.

### Devices

LAC-34 delivery can use devices such as a dermal patch or an injector. The patch can deliver LAC-34 as the free base or as a pharmaceutically acceptable salt, or an optically active isomer thereof, into the skin by diffusion or by using an active delivery system, such as for example iontophoresis or sonophoresis. Injectors can have a needle or be needleless. The concentration of LAC-34 used in dermal patches will be similar to the doses of lidocaine, presently used in Lidoderm® patches, which is 5 percent. Due to the low tissue toxicity of LAC-34, higher concentrations than 5 percent, such as up to 30 percent may also prove to be useful in a patch formulation containing LAC-34 or an isomer thereof. Dermal or transdermal patches may be applied one or more times daily. Due to the extreme potency of LAC-34, the concentration of LAC-34 in a patch may be as low as 0.1 percent. Due to the long duration of action of RS—, R— and S-LAC-34 free base, when located intradermally, intradermal patches may not always be applied daily, but may administered more seldom than once daily, such as for example every two or every seven days. The formulations of LAC-34 may be used in dermal patches and may contain LAC-34 in the form of free base or a pharmaceutically acceptable salt. Numerous types, sizes and forms of patches are available commercially (ex Teikoku Seiyaku Co., Ltd., Sanbonmatsu, Kagawa 769-2695, Japan) and can be used for the intradermal delivery of LAC-34 as the free base or a salt form or an isomers of LAC-34 as the free base or a salt form.

The total dose of LAC-34 delivered into the skin by a patch will depend on the quality of the skin, the size of the patch, the concentration of LAC-34 in the patch and the duration of the exposure of the patient to said patch. Needleless injectors and regular syringes may be useful devices for the administration of LAC-34 in water-soluble forms. Needleless devices will have obvious advantages, in selected patients, such as for example in children, and may be used in connection with dermal pain (insect bites, etc) or in preparation for expectedly painful injections with a regular syringe or in preparation for surgical procedures.

Parenteral injections of formulations containing LAC-34 are expected to have therapeutic effects on interneurons in the substantia gelatinosa of the dorsal spinal cord and other applicable biophases for this compound.

In general, the compositions of the present invention are prepared by dissolving the active ingredient, LAC-34, as a racemate or as a single isomer, preferably as a free base in a pharmaceutically acceptable volatile solvent, such as diluted DMSO, or a mixture of such solvents, at room temperature, and adding an anti-solvent, such as water, to the solution. Optional ingredients such as above mentioned permeation enhancers, solubilizers, plasticizers, propellants (in the case of aerosols) and capsaicinoids, may be added before or after the anti-solvent. Specific information regarding the manufacturing of various compositions of formulations is given in the following examples.

### Example 1

### Development of Analytical Method

HPLC analytical methodology was developed, using LAC-34 free base at room temperature (21-23° C.). A liquid's ability to dissolve LAC-34 was screened by adding a known mass of LAC-34 to a known volume/mass of liquid. Saturation solubility was measured by adding an excess of drug substance and allowing the suspension to equilibrate while stirring for 2-3 days. Four solutions of different concentrations of LAC-34 (8.1 mcg/mL to 0.98 mg/mL) plus a blank were typically tested in triplicate. Excellent linearity of the recovery was obtained over three orders of magnitude in LAC-34 concentrations and the relative standard deviations of peak areas for all LAC-34 solutions were less than 0.5%. The blanks assayed before and after LAC-34 solutions showed no significant drug level, which demonstrated that there was no carry-over.

### Example 2

Various excipients were tested for their capability to dissolve LAC-34, free base. Some results from these screening tests are shown in Table 2.

TABLE 2
Solubility (mg/mL) of LAC-34 free base at room temperature

| | Solubility |
|---|---|
| Excipient | of LAC-34 |
| Polar | |
| Ethanol | >86 |
| Isopropyl alcohol | >79 |
| Propylene glycol | >76 |
| Benzyl alcohol | >100 |
| Propylene carbonate | >78 |
| Hexylene glycol | >75 |
| Semi-polar/non-polar | |
| Dibutyl adipate | >79 |
| Isopropyl myristate | >78 |
| Isopropyl palmitate | 50-100 |
| Mineral oil | <50 |

With the exception of mineral oil, LAC-34 could be dissolved in high concentrations in all the solvents tested. LAC-34 has a yellowish-brown color when it exists as a glass and solutions in Table 2 were yellowish-brown.

When tested at 4° C., 20% LAC-34 in benzyl alcohol was a solution, 5% LAC-34 in propylene glycol was a gel, 5% LAC-34 in isopropyl myristate was a suspension, 5% LAC-34 in isopropyl palmitate was a solid, 5% LAC-34 in isopropyl alcohol was a suspension and cetyl alcohol was a solid (melting point=49.3° C.)

### Example 3

### Tests of Saturation Solubility in Selected Solvents

The saturation solubility of LAC-34 was measured in capric/caprylic triglycerides (CCT), dimethylsulfoxide (DMSO), ethanol, isopropanol, isopropyl myristate (IPM), pentane, propylene glycol (PG), and water. CCT and IPM were selected for further work since they can function as emollients and oily vehicles in creams. DMSO is a solvent and a penetration enhancer. Ethanol and isopropanol are volatile solvents and rapid evaporation of a solvent like ethanol and isopropyl alcohol may be important for minimizing therapeutic onset time. Propylene glycol has multiple functions in dermal formulations, the most important of which are solubilization and penetration enhancement. Some results from the saturation studies with LAC-34 are shown in Table 3.

The analytical HPLC method described in Example 1 was used. For the determination of water solubility, the injection volumes of aqueous filtrate equilibrated with water were kept at 50 μL since previous tests using spectrophotometric methodology had demonstrated extremely low water solubility.

TABLE 3
Saturation solubility (mg/mL) of LAC-34 free base
in key solvents for dermal drug delivery.

| | LAC-34 Solubility |
|---|---|
| Solvents | (mg/mL) |
| Capric/caprylic triglycerides (CCT) | 195 |
| Dimethylsulfoxide (DMSO) | 215 |
| Ethanol | >2,000 |
| Isopropanol | >526, <2,000 |
| Isopropyl myristate (IPM) | 184 |
| Pentane | 150* |
| Propylene glycol (PG) | 149 |
| Water | 0.005 |

*Pentane evaporates rapidly and is difficult to weigh. Therefore, this value is uncertain.

All of the solvents, with the exception of water, were able to dissolve large quantities of LAC-34. LAC-34, as the free base, was very soluble also in DMSO. Both IPM and CCT, which are semi-polar emollient oils, had also high solubilization capacity for LAC-34. Propylene glycol (PG), which has multiple actions for dermal drug delivery (see Table 1), was also able to solubilize significant quantities of LAC-34. Ethanol had a very high capacity to dissolve LAC-34 and was never saturated. The value for ethanol in Table 3 was obtained by adding 0.25 g of ethanol to 0.498 g of LAC-34, which rapidly resulted in a homogeneous solution. After observing this phenomenon with ethanol, 0.125 g of isopropanol was added to 0.25 g of LAC-34. Surprisingly, isopropanol was not able to solubilize the drug until the isopropanol level was 0.225 g. This was approximately 526 mg/mL.

It was concluded that opportunities exist to create super-saturated solutions on the skin by using crystallization inhibitors, such as for example polyvinyl pyrrolidone (Povidone, Merck Index 14^{th} Ed., 7697).

### Example 4

### Tests of Anti-Solvents/Binary Solvent Mixtures

Due to the very high solubility of LAC-34 free base in numerous volatile solvents, it was determined to be necessary to find and add an anti-solvent in order to reach saturation of the solutions of LAC-34. The efficacy of water is an anti-solvent for LAC-34 free base was investigated in the present studies. Table 4 shows the impact of water on solubility in some important solvents.

TABLE 4
Solubility of LAC-34 (in mg/mL) in mixtures of
solvent and water.

| % Water | PG | Ethanol | DMSO |
|---|---|---|---|
| 0 | 149 | >2000 | 215 |
| 1 | 139 | — | — |
| 2.5 | 104 | — | — |
| 5 | 63 | — | ≧100** |
| 10 | 29 | — | — |
| 15 | — | 63* | — |
| 20 | 8.7 | 33* | — |
| 30 | — | 7.0* | — |
| 50 | 0.37 | — | 1.7 |
| 75 | 0.075 | — | — |
| 90 | — | — | 0.45 |

*Titrated from an ethanol/LAC-34 solution with water
**Previous tests

As shown in Table 4, water was an excellent anti-solvent for LAC-34 in various solvents. This surprising finding was considered to be important since water is an endogenous molecule found in all mammals and additional toxicity testing of synthetic anti-solvents was therefore not needed. The decreased solubility is not merely due to the decreased concentration of the solvents, as is obvious from Tables 2 and 3 and from the very significant decrease of the solubility when small amounts of water is added, as shown in Table 4. Thus, water did not merely dilute the solvents, but water acted as an anti-solvent for the LAC-34 molecule when dissolved in the investigated excipients. It should be noted that an anti-solvent is a solvent in which the active pharmaceutical ingredient is insoluble and adding anti-solvent to a drug formulation should not be confused with adding water to a formulation when the active pharmaceutical ingredient is soluble in water. As an example, while developing transdermal formulations for a series of NSAIDs and antihistamines, Toppo (U.S. Pat. No. 5,985,860) added water as a co-solvent to reduce the high viscosity of his formulations, which was possible since all the NSAIDs and antihistamines used by Toppo had to be water-soluble.

### Example 5

### Tests of Anti-Solvents/Ternary Solvent Mixtures

Laboratory studies have now demonstrated the compatibility of combinations of solvents with water as an anti-solvent. Studies have demonstrated that a vehicle for LAC-34 could contain a solvent/penetration enhancer (e.g., propylene glycol, PG), water or buffer, plus a volatile solvent to create a saturated/super-saturated solution on the skin's surface. Therefore, several solutions were made where PG would be saturated or almost saturated with LAC-34 (i.e., −15% w/w). A quantity of ethanol was added to act at the volatile solvent. Each solution was titrated with water until it became cloudy. As with other titrations with water, the LAC-34 appeared to come out of solution as a liquid (oil) rather than a solid. Test results are summarized in Table 5.

TABLE 5
Ternary solubility studies with LAC-34.

| | | | Mass of | Saturation |
|---|---|---|---|---|
| Mass of | Mass of | Mass of | water | Concentration |
| LAC-34 | PG* | Ethanol | titrated | of LAC-34 |
| (g) | (g) | (g) | (g) | (% w/w) |
| 0.150 | 1.0 | 0.5 | 0.317 | 7.6 |
| 0.150 | 1.0 | 1.0 | 0.601 | 5.4 |
| 0.200 | 1.0 | 3.0 | 1.880 | 3.3 |

PG* = propylene glycol

The larger the amount of ethanol, the higher the amount of water was needed before LAC-34 precipitated. These results followed the same pattern observed in the binary systems: the anti-solvent (water) suppressed LAC-34 solubility and having more ethanol in the formulation necessitated more of the anti-solvent (water) to be incorporation before saturation. Therefore, there did not appear to be a “three-factor” interaction between PG, ethanol, and water. When prototype formulations are prepared, the ranges of water (or buffer) and solvents can be narrowed and varied using a mixture design to optimize LAC-34 solubility and stability in the solution.

Thus, the solubility of 150 mg of LAC-34 free base in a mixture of 1 gram of propylene glycol and 1 gram of ethanol was dramatically decreased to 5.4% (w/w) by the presence of 0.6 ml of the anti-solvent (water).

### Example 6

Aqueous and non-aqueous solutions.

TABLE 6
Compositions of solutions of LAC-34 and the
optically active isomers thereof for dermal and topical
applications

| | Percent w/w of |
|---|---|
| Component | Formulation |
| RS, R- or S-LAC-34 (base) | 0.1 to 30.0 |
| Ethanol | 5.0 to 25.0 |
| Propylene glycol | 10.0 to 75.0 |
| Antioxidants¹ | 0.01 to 0.1 |
| Preservatives² | 0.01 to 0.5 |
| Penetration enhancers³ | 0.01 to 50.0 |
| Anti-solvent water/buffer⁴ | 1.0 to 30.0 |

¹Examples: BHA, BHT
²Examples: methyl paraben, ethyl paraben, propyl paraben, imidurea
³Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), oleic acid.
One or more of the excipients marked 1 through 4 in Table 6 may not be needed - as will be determined in clinical tests - and if so, can be excluded.

The solutions will preferably contain from 0.1 percent to 30 percent of LAC-34, or an optically active isomer thereof, in its free base form. Pharmaceutically acceptable salts of LAC-34 may be used and are pharmacologically less active than the free base, which is therefore preferred. Some of the excipients may not be needed when low concentrations of LAC-34 are used. Preservatives will not be needed if single-dose vials are used. The solutions described here may be directly applied to the skin or may be used in patches.

Capsaicin (0.01% to 1.0%; w/w) can be included in these solution formulations, as well as in all other formulations of racemic or isomeric LAC-34, to obtain prolonged relief from pain and in particular from neuropathic pain. The racemic compound LAC-34 or an isomer thereof will inhibit or decrease the initial pain caused by capsaicin. Other capsaicinoids, such as dihydrocapsaicin, nordihydrocapsaicin, homocapsaicin homodihydrocapsaicin can also be used in analogous amounts, as can the partial capsaicin receptor antagonist capsazepine and other compounds that directly increase the inflow of calcium through the neuron membranes or indirectly influence ionic flow through neuron membranes through activation of the vanilloid subtype-1 receptor (such as for example resiniferatoxin).

### Example 7

### Methods of Manufacture: Solutions for Dermal and Topical Application

Dissolve LAC-34 in the solvent where it has the highest solubility (e.g., ethanol or isopropyl alcohol). Add additional solvents with preservatives, penetration enhancers, and antioxidants; then mix until well combined. Add water with buffers as needed. Mix until well combined then pack into appropriate container/closure system.

The racemic form of LAC-34 or an optically active isomer thereof can be used. The free base or a suitable salt form can be used. The solutions will preferably contain from 0.1% to 30% of 2-[2-(N-phenyl-N2-indanyl)aminoethyl]piperidine (LAC-34), or an optically active isomer thereof, in its free base form or as a pharmaceutically acceptable salt.

### Example 8

### Aqueous or Non-Aqueous Gels

A dermal gel formulation of LAC-34 or an isomer thereof, in its free base form, which is preferred, or as a pharmaceutically acceptable salt, can contain propylene glycol, ethanol, a polymer, and optionally another penetration enhancer and water or buffer as antisolvent. Since the amount of water in the formulation may be small, polymers that can gel non-aqueous system such as hydroxypropylcellulose (HPC) or hydroxyethylcellulose (HEC) can be used. In experiments with HEC, this excipient was found not only to ably gel the systems, but surprisingly, HEC was also found to be able to stabilize supersaturated solutions of LAC-34 or an isomer thereof on the skin's surface. Self-occlusion of gels can also be obtained by including methylcellulose as an excipient. As persons skilled in the art will realize, other topically acceptable gelling agents may also prove to be useful in gels containing LAC-34 or an isomer thereof in its free base form or as a pharmaceutically acceptable salt. Gels containing LAC-34 may be applied directly to the skin or may be used in patches.

TABLE 7
Examples of gels containing LAC-34 base for
dermal applications.

| | Percent w/w of |
|---|---|
| Component | Formulation |
| RS-, R- or S-LAC-34 | 0.1 to 30.0 |
| Ethanol | 5.0 to 25.0 |
| Propylene glycol | 50.0 to 75.0 |
| Polymer¹ | 0.5 to 3.0 |
| Antioxidants² | 0.01 to 0.3 |
| Preservatives³ | 0.01 to 0.5 |
| Penetration enhancers⁴ | 0.01 to 20.0 |
| Water/buffer | q.s. |

¹Examples: Hydroxypropylcellulose, hydroxyethylcellulose, methylcellulose, carbomer
²Examples: BHA, BHT
³Examples: Methyl paraben, ethyl paraben, propyl paraben, imidurea
⁴Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid.
One or more of the excipients marked 2 through 4 in Table 7 may not be needed - as will be determined in clinical tests - and if so, can be excluded.

The gels will preferably contain from 0.1 percent to 30 percent of LAC-34, or an optically active isomer thereof, in its free base form or as a pharmaceutically acceptable salt. Some of the excipients in Table 7 may not be needed when low concentrations of LAC-34 are used. Preservatives will not be needed if single-dose units are used.

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin homodihydrocapsaicin can also be used in analogous amounts, as can the partial capsaicin receptor antagonist capsazepine and other compounds that directly or indirectly influence ionic flow in neuron membranes.

### Example 9

### Methods of Manufacture: Gels

Dissolve LAC-34 or an optically active isomer thereof, in its free base form or as a pharmaceutically acceptable salt in the solvent where it has the highest solubility (e.g., ethanol or isopropyl alcohol). Add additional solvents with preservatives, penetration enhancers, and antioxidants then mix until well combined. Disperse polymer under appropriate conditions recommended by the manufacturer. Add water with buffers (if required) and neutralizing bases as needed. Mix until well combined and thickened then pack into appropriate container/closure system.

The racemic form of LAC-34 or an optically active isomer thereof can be used. The free base or a suitable salt form can be used. The gels will preferably contain from 0.1% to 30% of LAC-34, or an optically active isomer thereof, in its free base form or as a pharmaceutically acceptable salt.

Gels containing LAC-34 are intended for use on skin where patches are not preferred or cannot be used, such as for example by diabetic patients who most often express neuropathies on feet and/or hands. Gels may also be useful for the treatment of trigeminal neuropathies, which are located in the face, where the use of patches is not preferred.

### Example 10

### Emollient Creams and Lotions

The surprisingly high solubility of LAC-34 in its free base form in emollient oils was exploited to develop elegant cream formulations. When the oil-phase was approximately 55%, a 10% LAC-34 cream could be made with isopropyl myristate or capric/caprylic triglycerides. If needed, a penetration enhancer and other excipients could also be included.

TABLE 8
Creams containing LAC-34 base for dermal
applications:

| | Percent w/w of |
|---|---|
| Component | Formulation |
| RS-, R- or S-LAC-34 | 0.1 to 30.0 |
| Capric/Caprylic | 0.01 to 50.0 |
| Triglycerides¹ | |
| Isopropyl myristate² | 0.01 to 50.0 |
| Propylene glycol³ | 0.01 to 25.0 |
| Surfactant⁴ | 1.0 to 5.0 |
| Polymer⁵ | 0.5 to 3.0 |
| Antioxidants⁶ | 0.01 to 0.3 |
| Preservatives⁷ | 0.01 to 0.5 |
| Penetration enhancers⁸ | 0.01 to 30.0 |
| Anti-solvent water/buffer | 1.0 to 30.0 |

⁴Examples: sorbitan monolaurate, glycerol monolaurate, propylene glycol monolaurate, polysorbate 20, steareth-20
⁵Examples: Hydroxypropylcellulose, hydroxyethylcellulose, carbomer, xanthan gum
⁶Examples: BHA, BHT
⁷Examples: Methyl paraben, ethyl paraben, propyl paraben, imidurea
⁸Examples: DMSO, diethylene glycol monoethyl ether, n-decyl methyl sulfoxide, dimethylacetamide, laurocapram (Azone ®), dimethylformamide, N-methyl-2-pyrrolidine (Pharmasolve ®), sucrose monooleate and oleic acid.

One or more of the excipients marked 1, 2, 3, 6, 7, or 8 in Table 8 may not be needed—as will be determined in clinical tests—and if so, can be excluded.

The emollient creams or lotions will preferably contain from 0.1 percent to 30 percent of LAC-34, or an optically active isomer thereof, in its free base form or as a pharmaceutically acceptable salt. Some of the excipients in Table 8 may not be needed when low concentrations of LAC-34 are used. Preservatives will not be needed if single-dose units are used.

Capsaicin (0.01% to 1.0%; w/w) can be included in these formulations to obtain prolonged relief from neuropathic pain. The compound LAC-34 will inhibit or decrease the initial pain caused by capsaicin. Other capsaicinoids, such as dihydrocapsaicin, nordihydrocapsaicin, homocapsaicin and homodihydrocapsaicin can also be used in analogous amounts, as can the partial capsaicin receptor antagonist capsazepine and other compounds that directly or indirectly influence ionic flow in neuron membranes.

Emollient creams and lotions containing LAC-34 are intended for use on skin where patches are not preferred, such as for example by diabetic patients who most often express neuropathies on feet and/or hands. Emollient creams and lotions may also be useful for the treatment of trigeminal neuropathies in the face.

Creams and lotions containing LAC-34 may be applied directly to the skin or may be used in patches.

### Example 11

### Methods of Manufacture: Creams

Combine oils, semi-polar oils, antioxidants, penetration enhancers, and surfactants and heat to 60-75° C. (Phase A). Combine polar solvents, preservatives, water, buffer salts, and polymer and heat to 60-75° C. (Phase B). Dissolve LAC-34 in Phase A then immediately add Phase A to Phase B with rapid stirring. High-shear mixing for a specified time may also be required. Cool the mixture while stirring to 30-50° C. If the polymer requires a basic substance to thicken (e.g., sodium hydroxide or triethanolamine), add the base. Once product is sufficiently thickened and cool (≦35° C.), stop stirring and begin packing the product into the appropriate container closure system.

The racemic form of LAC-34 or an optically active isomer thereof can be used. The free base or a suitable salt form can be used. The emollient creams or lotions will preferably contain from 0.1 percent to 30 percent of LAC-34, or an optically active isomer thereof, in its free base form or as a pharmaceutically acceptable salt.

### Example 12

### Ointments for Dermal, Rectal and Vaginal Application

While the unexpectedly high solubility of LAC-34 base in practically all excipients (not water) complicated the formulation of dermal solutions, gels and cream containing LAC-34, the high solubility of LAC-34 in “oily” excipients like isopropyl myristate and mineral oil can also bee seen as an advantage since such excipients tend to be non-irritating and therefore suited to an emollient cream formulation for extended use and for ointments (i.e., hemorrhoids, shingles, etc.).

TABLE 9
Ointments (and non-irritant creams) containing LAC-
34 for dermal and rectal use.

| | Percent w/w of |
|---|---|
| Component | Formulation |
| RS-, R-, or S-LAC-34 | 1.0 to 30.0 |
| Propylene glycol | 2.5 to 10.0 |
| Microcrystalline wax | 5.0 to 30.0 |
| White wax (bees wax)¹ | 0.01 to 20.0 |
| Surfactants² | 0.5 to 5.0 |
| Preservatives³ | 0.01 to 0.5 |
| Antioxidants⁴ | 0.01 to 0.1 |
| Penetration enhancer⁵ | 0.01 to 20.0 |
| Mineral oil | q.s. |

¹Examples: bees wax
²Examples: sorbitan monolaurate, glycerol monolaurate, propylene glycol monolaurate, polysorbate 20, steareth-20
³Examples: Methyl paraben, ethyl paraben, propyl paraben, imidurea
⁴Examples: BHA, BHT
⁵Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid
One or more of the excipients marked 1, 2, 3, 4 and 5 in Table 9 may not be needed - as will be determined in clinical tests - and if so, can be excluded.

The ointments and non-irritant creams will preferably contain from 0.1 percent to 30 percent of LAC-34, or an optically active isomer thereof, in its free base form or as a pharmaceutically acceptable salt. Some of the excipients in Table 9 may not be needed when low concentrations of LAC-34 are used, as will be determined in future clinical tests. Preservatives will not be needed if single-dose units are used.

Ointments and lotions containing LAC-34 may be applied directly to the skin or may be used in patches.

Capsaicin (0.01% to 1.0%; w/w) can be included in these formulations to obtain prolonged relief from neuropathic pain.

The compound LAC-34 will inhibit or decrease the initial pain caused by capsaicin. Other capsaicinoids, such as dihydrocapsaicin, nordihydrocapsaicin, homocapsaicin homodihydrocapsaicin can also be used in analogous amounts, as can the partial capsaicin receptor antagonist capsazepine and other compounds that directly or indirectly influence ionic flow in neuron membranes.

### Example 13

### Methods of Manufacture: Ointments

Combine waxes, oils, and semi-polar oils, and antioxidants and heat to 60-75° C. (Phase A). Combine LAC-34 with solvent (e.g., propylene glycol) and penetration enhancer (if used) and heat to 60-75° C. (Phase B). Add Phase B to Phase A and mix under high shear while cooling. Once ointment thickens, stop high shear and mix with low shear until product is sufficiently cool (≦35° C.), stop stirring and pack the product into the appropriate container closure system.

The racemic form of LAC-34 or an optically active isomer thereof can be used. The free base or a suitable salt form can be used.

The ointments and non-irritant creams will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

### Example 14

### Suppositories for Rectal or Vaginal Applications

Suppositories are useful for the treatment of vaginal and rectal pain, particularly hemorrhoidal pain.

TABLE 10
Suppository formulations containing LAC-34 base
for rectal or vaginal applications.

| | Percent w/w of |
|---|---|
| Component | Formulation |
| RS-, R- or S-LAC-34 | 0.1 to 20.0 |
| Antioxidants¹ | 0.01 to 0.3 |
| Preservatives² | 0.01 to 0.5 |
| Penetration enhancers³ | 0.01 to 10.0 |
| Suppository matrix⁴ | q.s. |

¹Examples: BHA, BHT
²Examples: Methyl paraben, ethyl paraben, propyl paraben, imidurea
³Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid
⁴Polyethylene glycol (molecular weights 200 to 20,000), hydrogenated vegetable oil (e.g., Wecobee ® S, Wecobee ® FS
One or more of the excipients marked 1 through 3 in Table 10 may not be needed - as will be determined in future clinical tests - and if so, can be excluded.

The suppositories will preferably contain from 0.1% to 30% of LAC-34, or an optically active isomer thereof, in its free base form or as a pharmaceutically acceptable salt. Some of the excipients may not be needed when low concentrations of LAC-34 are used. Preservatives will not be needed if single-dose units are used.

### Example 15

### Methods of Manufacture: Suppositories

Melt suppository matrix material (i.e., polyethylene glycol or partially hydrogenated vegetable oil) then dissolve LAC-34 in the melt. Fill melt into mold, cool, and un-mold. Pack suppositories in appropriate container/closure system.

The racemic form of LAC-34 or an optically active isomer thereof can be used in concentrations from about 0.1 percent to about 20 percent. The free base or a suitable salt form can be used.

The suppositories will preferably contain from 0.1 percent to 30 percent of LAC-34, or an optically active isomer thereof, preferably in its free base form but pharmaceutically acceptable can also be used.

### Example 16

### Aerosol Sprays (Pump or Propellant Driven)

In addition to the solubility experiments shown in Table 3, the solubility of LAC-34 (base) was also investigated using pentane since this is an example of low molecular weight hydrocarbons, commonly used with a GRAS aerosol propellant such as butane. The result is shown below. The very high solubility of LAC-34 in pentane indicates that hydrocarbon aerosol propellants such as butane will be useful for drug delivery to the skin by aerosol.

Aerosol sprays (pump or propellant driven) of LAC-34 are intended for use where on skin with pronounced hyperalgesia, such as skin expressing shingles, where LAC-34 will serve the double purpose of acute inhibition of pain and prevention of the development of post-herpetic neuralgias. Aerosol sprays of LAC-34 will also be useful where patches are not preferred, such as for example for diabetic patients who most often express neuropathies on feet and/or hands. Aerosol sprays may also be useful for the treatment of trigeminal neuropathies in the face, where the use of patches are not preferred.

TABLE 11
Saturation solubility of LAC-34 in pentane.

| Solvent | LAC-34 Solubility, mg/mL |
|---|---|
| Pentane | 150* |

*Pentane evaporates rapidly and is difficult to weigh. Therefore, the solubility information may not be exact.

Since LAC-34 is very soluble in both volatile solvents and low molecular weight hydrocarbons, aerosol formulations of LAC-34 are possible. The aerosol can contain a volatile solvent and propellant along with a non-volatile solvent plus one or more penetration enhancers. Numerous propellants are known to those skilled in the art and can be used with LAC-34 in aerosol sprays. When ethanol is the volatile solvent, a pump spray will be required to create an aerosol. When butane is used, the container/closure system can be pressurized and only a metering valve is required to create a useful aerosol administration system.

TABLE 12
Examples of aerosol formulations of LAC-34
(propellant driven or pump-driven)

| Component | % w/w of Formulation |
|---|---|
| RS-, R- or S-LAC-34 | 0.2 to 30.0 |
| Propylene glycol¹ | 0.01 to 40.0 |
| Surfactant² | 0.05 to 5.0 |
| Polymer³ | 0.01 to 3.0 |
| Antioxidants⁴ | 0.01 to 0.3 |
| Preservatives⁵ | 0.01 to 0.5 |
| Propellants⁶ | 0.01 to 40.0 |
| Penetration enhancers⁷ | 0.01 to 50.0 |
| Water/buffer | q.s. |

²Examples: sorbitan monolaurate, glycerol monolaurate, propylene glycol monolaurate, polysorbate 20, steareth-20
³Examples: Hydroxypropylcellulose, hydroxyethylcellulose, carbomer
⁴Examples: BHA, BHT
⁵Examples: Methyl paraben, ethyl paraben, propyl paraben, imidurea
⁶Examples: Butane, isobutane, propane, hydrofluoralkane (134a, 227)
⁷Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid
One or more of the excipients marked 1, 3, 4, 5, 6 and 7 in Table 12 may not be needed and if so, can be excluded.

The aerosols will preferably contain from 0.1% to 30% of LAC-34, or an optically active isomer thereof, in its free base form or as a pharmaceutically acceptable salt. Some of the excipients may not be needed when low concentrations of LAC-34 are used. Preservatives will not be needed if single-dose units are used

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin and homodihydrocapsaicin can also be used in analogous amounts, as can the partial capsaicin receptor antagonist capsazepine and other compounds that directly or indirectly influence ionic flow in neuron membranes.

### Example 17

### Methods of Manufacture: Aerosols

If the product is a pump spray, prepare solution as described in the solutions/gels section and fill into the appropriate container/closure system with spray pump.

If the product is a propellant-driven spray, dissolve LAC-34 in liquefied propellant and add appropriate solvents, penetration enhancers, and surfactants/polymers. Mix until homogeneous then fill under pressure into the appropriate container/closure system. Alternatively, LAC-34 plus solvents, surfactants/polymers, and penetration enhancers can be combined at room temperature/pressure then filled into an aerosol canister. After crimping the metering valve in place, the propellant can be pressure-filled into the container.

Alternatively, the acrylic polymer or copolymer is dissolved in the chosen vehicle; the active ingredient together with the permeation enhancer is dissolved in the solution, the plasticizer and remaining ingredients are added, the cans are filled, the liquefied propellant is added and the metering valve is crimped in place.

The racemic form of LAC-34 or an optically active isomer thereof can be used for aerosols. The free base or a suitable salt form can be used.

The aerosols will preferably contain from 0.1% to 30% of LAC-34, or an optically active isomer thereof, in its free base form or as a pharmaceutically acceptable salt, preferably as the free base.

### Example 18

### Parenteral Formulations for LAC-34, Useful for Prevention and Treatment of Nociceptive Pain or Neuropathic Pain After Injection

Excipients included in a solution for injections of LAC-34×2HCl are shown in Table 13.

TABLE 13
Formulations of LAC-34 for injections

| Component | % w/w of Formulation |
|---|---|
| RS-, R- or S-LAC-34 × 2HCl | 0.1 to 10.0 |
| Chelating agents¹ | 0.01 to 0.3 |
| Antioxidants² | 0.01 to 0.3 |
| Preservatives³ | 0.01 to 0.5 |
| Vasoconstrictors⁴ | 0.01 to 0.05 |
| Water/buffer | q.s. |

¹Examples: Disodium edetate, citric acid
²Examples: BHA, BHT, ascorbic acid
³Examples: Methyl paraben, ethyl paraben, propyl paraben, imidurea
⁴Examples: Epinephrine, phenylephrine
One or more of the excipients marked 1, 2, 3 or 4 in Table 13 may not be needed and if so, can be excluded.

The solutions for parenteral use will preferably contain from 0.1 percent to 10 percent of LAC-34, or an optically active isomer thereof, preferably as a water-soluble, pharmaceutically acceptable salt. Some of the excipients may not be needed when low concentrations of LAC-34 are used. Preservatives will not be needed if single-dose units are used.

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin and homodihydrocapsaicin can also be used in analogous amounts, as can the partial capsaicin receptor antagonist capsazepine and other compounds that directly or indirectly influence ionic flow in neuron membranes.

### Example 19

### Methods of Manufacture: Parenteral Solutions

Combine ingredients, including active, in an appropriate vessel and mix until homogeneous. Aseptically filter and fill the solution into the appropriate container/closure system. Terminal sterilization is also an option in the drug product preparation if required.

The racemic form of LAC-34 or an optically active isomer thereof can be used. A water-soluble salt form is preferably used.

The solutions for parenteral use will preferably contain from 0.1 percent to 30 percent of 2-[2-(N-phenyl-N-2-indanyl)aminoethyl]piperidine, or an optically active isomer thereof, preferably as a water-soluble salt.

### Example 20

### Inhibition of Voltage-Gated Sodium Channels by LAC-34

The inhibitory effects of LAC-34 on batrachotoxin-sensitive Na⁺ channel binding sites were tested using binding assays. The inhibitory effects of LAC-34 on human NaV1.3 and NaV1.5 channels were tested using electrophysiological methodology on stably expressed in mammalian cells (HEK-293). Inhibition of NaV1.8 channels were tested using electro-physiological methodology on dorsal root ganglia (DRG neurons)

### A. Batrachotoxin-Sensitive Na Channel (Site 2)

Inhibition of batrachotoxin-sensitive sodium channels refers to antinociceptive (local anesthetic) activity (Nishizava et al, 1988 which publication is hereby included by reference.)

### Methodology

Na⁺ channels from rat cerebral cortex were used in receptor binding studies. [³H]batrachotoxin (10 nM) was used as the specific ligand and veratridine as non-specific ligand. Incubation was 60 min/22° C. and the method of detection was scintillation counting. The specific ligand binding to the receptors was defined as the difference between the total binding and the nonspecific binding determined in the presence of an excess of unlabelled ligand. The IC₅₀ values (concentration causing a half-maximal inhibition of control specific binding) and Hill coefficients (n_{H}) were determined by non-linear regression analysis of the competition curves using Hill equation curve fitting. LAC-34 and lidocaine were tested in parallel. All tests were performed in duplicate at 12 and 10 concentrations of LAC-34 and lidocaine, respectively.

### Results

LAC 34: IC₅₀ was 2.47E-07M

Lidocaine: IC₅₀ was 5.25E-05M

### Conclusion

It was calculated that LAC 34 is approximately 210 times more potent than lidocaine as an inhibitor of batrachotoxin-sensitive Na-channels. This activity is usually referred to local anesthesia (prevention and treatment of nociception.)

### B. NaV1.3 Sodium Channel

Upregulated NaV1.3 channels are involved as neural pacemakers and mediate the expression of neuropathic pain (Mo, G. et al. 2011, which publication is hereby included by reference.)

### Methodology

Electrophysiological methodology was used and mammalian (HEK-293) cells, stably expressing NaV1.3 channels and superfused with vehicle control solution. The intracellular pipette contained solutions with the test article (LAC-34 or lidocaine.) A commercial patch clamp amplifier was used.

### Results

LAC 34: IC₅₀ was 4 μM

Lidocaine 70% inhibition at 2 mM

### Conclusion

It was calculated that LAC 34 is more than 200 times as potent as lidocaine in the inhibition of NaV1.3 channels.

### C. NaV1.5 Sodium Channel

The NaV1.5 sodium channel is considered to be involved in neuropathic pain since NaV1.5-inhibitors have been effectively used in the treatment of this disease (Lenkowski et al., 2004, which publication is hereby included by reference.)

### Methodology

The same methodology as previously used for tests on NaV1.3 channels was also used for NaV1.5 channels.

### Results

LAC 34: IC₅₀ was 0.6 μM

Lidocaine 90% inhibition at 2 mM

### Conclusion

It was calculated that LAC 34 is about 500 times more potent than lidocaine in the inhibition of NaV1.5 channels.

### D. NaV1.8 Sodium Channel

NaV1.8 is another voltage-sensitive sodium channel that is directly linked to neuropathic pain (Lai et al. 2002, which publication is hereby included by reference.)

### Methodology

The NaV1.8 currents were measured in dorsal root ganglia (DRG), using patch clamp technology.

### Results

LAC 34: IC₅₀ was 2 μM

Lidocaine Approximately 100% inhibition at 2 mM

### Conclusion

It was calculated that LAC 34 is about 200 times more potent than lidocaine in the inhibition of NaV1.8 channels.

### Example 21

### Pharmacological Comparison Between Dermal Anesthetic Activity (Dermal Residence Times) of the Free Base and Various Salt Forms of LAC-34

The purpose of this study was to determine the efficacy of the free base and pharmaceutically acceptable salt forms of LAC-34 as dermal anesthetics, following dermal application of the compounds to guinea pigs. The Duration of Anesthesia is directly related to the Dermal Residence Times of the test articles.

### Methods

Four groups of six male guinea pigs received a dose of 2.0 ml of a 5.0% solution of four test articles (LAC-34 base, LAC-34 mono-HCl salt, LAC-34 di-HCl salt and LAC-34 mesylate salt) that had been applied onto a two-inch square, 4-layer thick gauze pad on depilated dorsal skin of each guinea pig. Similarly, one group of guinea pigs received the vehicle, which consisted of DMSO/water and served as the control group. The gauze was occluded and the guinea pigs were wrapped for 30 min. Animals were assessed for initial pain reaction (anesthetic depth) at 15-minute intervals up to 3 hours and at 24 hours. An algesimeter, set at 10 g, was employed to assess the onset and depth of anesthesia using the pinprick method causing a twitch response or non-response. The “Average Anesthetic Score” was defined as number of probings not producing a dermal twitch response. Thus, a situation where there were no dermal twitch responses to six probings represent a maximal anesthetic effect and is denoted as an Average Anesthetic Score of 6. Duration of Anesthesia was in this study defined as the time from patch removal until the test scores have regressed to an average anesthetic score below 2.0. The Duration of Anesthesia in this type of experiments is directly related the duration of Dermal Residency for the test articles.

### Results

The Average Anesthetic Scores after patch removal are shown in Table 14, below.

TABLE 14
Average Anesthetic Scores after patch removal.

| Treatment | Time after Patch Removal (min) | | | | |
|---|---|---|---|---|---|
| Group | 15 | 30 | 60 | 90 | 180 |
| LAC-34 Free Base | 4.8 | 4.8 | 4.4 | 3.3 | 2.2 |
| LAC-34 Mono-HCl | 0.2 | 0.8 | 1.3 | 0.7 | 0.7 |
| LAC-34 Mesylate | 0.7 | 0.7 | 1.8 | 1.5 | 0.2 |
| Vehicle | 0.0 | 0.0 | 0.0 | 0.0 | 0.0 |

The results shown in the table 14, demonstrate a favorable effect of LAC-34 free base when compared with the salt forms tested here. The Duration of Anesthesia was longer for the free base (180 min) than for any of the salts 60 min) and the Maximal Depth of Anesthesia (4.8 for LAC-34 free base) was more favorable for the free base than for any of the salt forms (1.3 for LAC-34 mono-hydrochloride salt; 1.8 for LAC-34 mesylate salt.) The dermal anesthetic effects were fully reversible as evidenced by consistent Anesthetic Scores of zero when tested 24 hours after patch removals.

conclusion

The Duration of Dermal Anesthetic activity of LAC-34 free base was significantly more pronounced that that of any of the LAC-34 salt forms. However, the tested pharmaceutically acceptable salt forms were active, expressing various levels of activity. The Dermal Residence Times for the various forms of LAC-34 is directly related to the Duration of Anesthesia and was significantly longer for the free base than for any of the salt forms.

### Example 22

### Physicochemical Properties of LAC-34

Water solubility and dissociation constants (pKa) were determined for the lead compound LAC-34 and reference compounds.

### Methods

Water solubility and dissociation constants were determined for the free base of LAC-34 using standard spectrophotometric, HPLC and potentiometric methods.

### Results

TABLE 15
Water solubility and pKa for LAC-34 (free base) and
reference compounds.

| | Water | Dissociation |
|---|---|---|
| | Solubility | Constant |
| Compounds | (μg/ml) | (pKa) |
| LAC 34 | 5*) | 8.09 ± 0.23 |
| Lidocaine | 3390 | 8.82 ± 0.12 |
| Tetracaine | 356 | 8.51 ± 0.12 |

*)see Table 3

### Conclusion

A dissociation constant close to 8 is almost ideal for a compound of this type, but the low water solubility (and high solubility in solvents and propellants; see Tables 1 and 3) represented unusual challenges as formulations had to be made that avoided transdermal penetration of excipients in which LAC-34 was dissolved.

### Example 23

### Studies on Transdermal Penetration Across Intact Human Skin

The indanylamines of the present application are highly soluble in DMSO (dimethylsulfoxide) and ethanol and numerous other solvents. It would therefore be a very simple task to make transdermal formulations of these compounds—dissolving the compounds in one or more tissue-penetrating solvents and applying the solution to the skin would result in transdermal penetration of the solvent(s) (containing the test article) across the skin, and the subsequent subdermal (subcutaneous) absorption of the test article by capillary blood vessels. To further improve the subdermal absorption and transport the active ingredient away from the skin, a vasodilator could be added, as was done by Toppo (U.S. Pat. No. 5,985,860), who added known vasodilators such as nicotinate and even castor oil that is known to release nitric oxide, which is the most potent vasodilator in the body.

However, the biophase (sites of therapeutic activity) for the indanylamines of the present invention is located in the layer of the skin that is called epidermis and to a lesser degree in the dermis. Therefore, special intradermal formulations had to be developed to allow for intradermal accumulation of the active compound in the epidermis. These formulations, though, must have the ability to force LAC-34 to cross the penetration barrier (stratum corneum) before said compound accumulates in the epidermis.

The present study was designed to determine the penetrability of LAC-34 across the skin when using a transdermal formulation (12A) and two intradermal formulations (19B containing an antisolvent and 20B without anti-solvent.)

### Method

C¹⁴-radioactive LAC-34 was synthesized by Eaglepicher Pharmaceutical Services, Lenexa, Kans., as a 10.4 nCi C¹⁴ powder of LAC-34 free base.

Healthy human skin was obtained from cosmetic surgery and while still containing living tissues, pieces of the donor skin were mounted in flow-through dissociation cells. Formulations containing C¹⁴-radioactive LAC-34 were applied on the outside of the skin and fluid from the subdermal/receptacle side of the dissociation chambers were analyzed for C¹⁴-LAC-34. The fluids on the receptacle side of the skin was continuously replaced in order to maintain the concentration gradient across the skin and thereby mimicking the in vivo situation, where subcutaneous deposits are continuously absorbed by capillary blood vessels.

In order to minimize the handling of the radioactive material, the excipients were initially prepared and mixed, whereupon radioactive LAC-34 was prepared and added to a concentration of 10 percent (w/w) of the final formulation.

The 12A-formulation was used as reference transdermal formulation and consisted of ten percent C¹⁴-labelled LAC-34 free base, dissolved in 100 percent DMSO. DMSO-formulations are considered to be a Golden Standard for transdermal delivery formulations and are used here as reference formulation since DMSO is known to cause transport across the skin of compounds that can be dissolved in this excipient. Other transdermal formulations—like those of Toppo (U.S. Pat. No. 5,985,860) cannot be used for LAC-34 since the Toppo formulations requires a water solubility of 1 mg/ml or more of the test article, which cannot be obtained with LAC-34 (see Table 3).

The 19B-formulation contained 60 percent isopropyl alcohol, 20 percent propylene glycol, 10 percent anti-solvent (water) and 10 percent C¹⁴-labelled LAC-34 free base.

The 20B-formulation was similar to the 19B-formulation, but did not contain any anti-solvent (water).

Each formulation was tested in five dissociation cells and mean values (MV)±standard errors (SEM) were calculated. Radioactivity was measured with a scintillation counter.

The hourly transport of radioactivity across the skin was calculated in percent of the amount of radioactivity that was tape-stripped from the outside of the skin.

Numerous formulations have been tested for their ability to transport LAC-34 across human skin. The results from tests of three such formulations (the transdermal formulation 12A and the intradermal formulations 19B (with an antisolvent) and Formulation 20B (without an antisolvent) are shown as examples herein.

### Results

As shown in Table 16, the DMSO-formulation (12A) transported a large amount—1.5 percent/hour—of the radioactive test article (LAC-34) across the skin into the receptacle side of the dissociation chamber after the start of the experiments. The transdermal transport of LAC-34 in the formulation 19B was significantly slower than the DMSO-formulation. Formulation 20B—without any anti-solvent—did not transport the radioactive compound across the skin.

TABLE 16
Transdermal transport of C¹⁴-labelled LAC-34 across
human skin.

| Formulation | Hourly subdermal recovery of |
|---|---|
| containing | C¹⁴-LAC-34, expressed as percent |
| C¹⁴-LAC-34 | (Mean values ± SEM) |
| 12A | 1.50 ± 0.13 |
| 19B | 0.56 ± 0.07 |
| 20B | 0.05 ± 0.01 |

The table shows the amount of radioactivity being transported across the skin and into the receptacle side of the dissociation chamber (“under the skin”), calculated as percent of the radioactivity in the skin strips.

### Conclusions

DMSO—a prototype transdermal formulation—transported LAC-34 across the skin and into the subdermal area at a rate of approximately 1.5 percent of the radioactivity/hour. The Formulation 19B transported C¹⁴-labelled LAC-34 at a rate of 0.56 percent/hour and Formulation 20B that did not contain an antisolvent was unable to transport C¹⁴-LAC-34 across human skin.

### Example 24

### Studies on Accumulation of LAC-34 in the Epidermis and Dermis

While Example 23 concerned transdermal transport of LAC-across human skin, the present Example 23 concerns transport of LAC-34 to the epidermis and dermis layers of the skin after application to the surface of the skin. The epidermis and the dermis are the layers of the skin where the thin afferent nerves and the nerve endings with the sodium channels are located. Thus, the present Example 23 concerns the transportation of LAC-34 to the biophase (site of action) of the drug. Numerous formulations have been tested for their ability to cause an accumulation of LAC-34 in the biophase of the drug (epidermis and dermis) and as examples, the results from tests of two such formulations (the transdermal formulation 12A and the intradermal formulation 19B are shown as examples herein.

### Methodology

The same diffusion cells as used in Example 22 were also used for the present Example 24 studies. The test formulations were prepared as described for Example 23, above. The present study also used human skin, and the test formulations, containing 10 percent radio-labeled LAC-34, were applied to the outside of the skin. At the termination of the each experiment, the deposits of radioactive LAC-34 in various layers of the skin were analyzed after separations of the dermal layers. Residual formulation was removed from the surface of the skin using three tape strips (D-Squame stripping disks.) The epidermis and dermis were isolated manually by blunt dissection, i.e. careful scraping with a spatula. The amount of radioactivity in the tape strips, the epidermis and the dermis were determined by use of a scintillation counter.

The compositions of Formulations 12A and 19B were described above (Example 23).

The formulations and the radioactive LAC-34 were prepared according to Example 23. The final transdermal formulation (12A) contained 90 percent of concentrated DMSO (w/w) and 10 percent (w/w) of radiolabeled LAC-34. The final intradermal formulation (19B) contained 60 percent isopropyl alcohol, 20 percent propylene glycol, 10 percent of the anti-solvent (water) and 10 percent radiolabeled LAC-34 (w/w).

### Results

The amount of radioactivity in the epidermis and dermis were calculated as percent of the radioactivity in the tape strips. The results shown in Table 17 represent Mean Values from five tests. SEM values (not shown in Table 17) were less than 10 percent of the Mean Values.

TABLE 17
The amount of LAC-34 (measured as C¹⁴-activity) in
percent of the initial radioactivity in the tape strips.

Recovery of C¹⁴-LAC-34 as percent
of concentration in tape strips

| Formulations | Epidermis | Dermis |
|---|---|---|
| 12A | 9.0 | 8.8 |
| 19B | 27.8 | 10.7 |

The amounts of radiolabeled LAC-34 in both the epidermis and the dermis were about 9 percent of the radioactivity in the tape strips when the DMSO formulation was tested, and were about 28 and 11 percent when the formulation 19B was tested.

### Conclusions

The DMSO formulation (12A) transported dissolved LAC-34 across the skin as shown in Example 22, and the relatively low concentration of radioactivity (9 percent) in the epidermis in the present study is believed to represent radioactivity “en route” through the skin.

The new formulation 19B caused a high concentration of radioactive LAC-34 to accumulate within the skin, particularly in the epidermis (approximately 28 percent of the tape strip concentration.) The composition of Formulation 19B is described above. When applied to the skin of patients, the volatile phase of Formulation 19B will evaporate and as shown in Table 4, the solubility of LAC-34 in the remaining excipients will rapidly decrease, thereby forcing the molecules of LAC-34, but not the solvent(s), to penetrate into the skin.

### Example 24

### Studies on Dermal Toxicity

It is well known by those skilled in the art that the risk for dermal toxicity (seen as erythema and/or edema in Draize tests) is extremely high after intradermal precipitation of compounds. The purpose of the present study was to determine dermal toxicity by formulations of LAC-34 in Hairless Guinea Pigs, which is a well known model for dermal irritation studies (Gad, 2007.)

### Method:

The present study was performed in vivo in hairless guinea pigs, where various formulations (25 μl) containing LAC-34 (10 percent) were applied to the surface of the skin.

Dermal toxicity was assessed as follows.

### Erythema Evaluation:

A standard Draize scale was used to assess erythema intensity.

0: No erythema;
1: Very slight erythema (barely perceptible);
2: Well-defined erythema;
3: Moderate to severe erythema;
4: Severe erythema (beet redness) to slight escar formations.

The assessments of erythema were performed at the following time points: 0, 1, 2, 4, 8, 24, 48 and 72 hours.

### Edema Evaluation:

A standard Draize scale was used to assess edema intensity.

0: No edema;
1: Very slight edema (barely perceptible);
2: Slight edema (edges of area well defined by definite raising);
3: Moderate edema (raised approximately 1 millimeter);
4: Severe edema (raised more than 1 millimeter and extending beyond area of exposure).

The assessments of edema were performed at the following time points: 0, 1, 2, 4, 8, 24 and 48 hours.

The compositions of Formulations 12A, 19B and 20B were as described above (Example 23).

The preparations of the formulations were described above (Example 7).

### Results and Conclusions:

DMSO 100 percent caused significant erythema and edema and the scores were even higher for Formulation 12A, consisting of 10 percent LAC-34 dissolved in 100 percent DMSO. Tissue irritation by DMSO is well known to those skilled in the art.

While several of the new formulations of LAC-34 caused dermal toxicity (erythema and/or edema), the formulations 19B and 20B, both containing 10 percent LAC-34, did not cause either erythema or edema.

It was surprising that Formulation 19B, containing 10 percent LAC-34, did not cause any erythema or any edema, although a considerable amount of the extremely potent compound LAC-34 was accumulating in the epidermis of the skin (see Example 23, above) and although LAC-34 is more than 200 times more potent than lidocaine.

The reason for showing the selected studies described herein, (Examples 22, 23 and 24) is to demonstrate the differences between simple formulations offering systemic drug absorption by transdermal transport and the far more complicated biophase-directed formulations of the present invention that are intended to make an accumulation of LAC-34 possible in the epidermis and dermis layers of the skin.

### Equivalents

Those skilled in the art will realize that liposome formulations containing LAC-34 and the isomers thereof may be clinically useful. There is a wide variety of liposomes available and using no more than routine experimentation, those skilled in art may develop clinically useful formulations containing LAC-34 and suitable liposome suspensions. All equivalents are intended to be encompassed in the scope of the present invention.

All doses indicated in this document refer to human doses. For use in animals, the doses may be the same as human doses or may be lower or higher, depending on the species and the reason for the treatment. The animal caretaker or a veterinarian will be able to determine the useful dose and concentration that is preferred to specific animals.

Doses higher or lower than indicated here may be useful in humans or in animals and all useful dose levels are intended to be encompassed in the scope of this invention.

What is claimed is:

1. An intradermal formulation for inducing relief of neuropathic pain, comprising a therapeutically effective amount of the compound 2-[2-(N-phenyl-N-2-indanyl)aminoethyl]piperidine, or an optically active isomer thereof or pharmaceutically acceptable salt thereof, dissolved in one or more solvents, at least one of which is volatile, in which said compound, isomer or salt is soluble, together with an anti-solvent in an amount sufficient to reduce the solubility of said compound, isomer or salt in said one or more solvents such that upon application of said formulation to the skin of a patient, said volatile solvent evaporates to form a saturated solution of said compound, isomer or salt, facilitating the penetration of said compound, isomer or salt into the skin and accumulation of said compound, isomer or salt in the epidermis and dermis of the skin in an amount effective for inducing relief of neuropathic pain.

2. The formulation according to claim 1 that contains from 0.1 percent to 30 percent of said compound, isomer or salt.

3. The formulation of claim 1, wherein said piperidine is in the form of its free base.

4. The intra-dermal formulation according to claim 1, wherein the said one or more solvents is a member selected from the group consisting of ethanol, dimethylsulfoxide, isopropanol, propylene carbonate, propylene glycol, decylmethylsulfoxide, N,N-dimethyl acetamide, 2-pyrrolidone, N,N-dimethyl formamide, 1-methyl-2-pyrrolidone, 5-methyl-2-pyrrolidone, 1,5-methyl-2-pyrrolidone, 1-ethyl-2-pyrrolidone, 2-pyrrolidone-5-carboxylic acid, oleic acid, laurocapram (azone), limonene, cineole, diethyl-m-toluamide (deet), sodium dodecylsulfate, trimethyl phosphine oxide, tetrahydrofurfuryl alcohol, glycerol, monolaurate, methyl oleate, and propylene glycol monolaurate, and mixtures thereof.

5. The intra-dermal formulation according to claim 2, wherein the said one or more solvents is a member selected from the group consisting of ethanol, dimethylsulfoxide, isopropanol, propylene carbonate, propylene glycol, decylmethylsulfoxide, N,N-dimethyl acetamide, 2-pyrrolidone, N,N-dimethyl formamide, 1-methyl-2-pyrrolidone, 5-methyl-2-pyrrolidone, 1,5-methyl-2-pyrrolidone, 1-ethyl-2-pyrrolidone, 2-pyrrolidone-5-carboxylic acid, oleic acid, laurocapram (azone), limonene, cineole, diethyl-m-toluamide (deet), sodium dodecylsulfate, trimethyl phosphine oxide, tetrahydrofurfuryl alcohol, glycerol, monolaurate, methyl oleate, and propylene glycol monolaurate, and mixtures thereof.

6. The intra-dermal formulation according to claim 1, further comprising one or more non-ionic surfactants selected from the group consisting of dimethylsulfoxide, ethanol, isopropanol, propylene glycol, glycerol monolaurate, propylene glycol monolaurate, sorbitan monolaurate, sorbitan sesquioleate, polysorbate 20, peg-40 stearate, steareth-20, poloxamer 185.

7. The intra-dermal formulation according to claim 2, further comprising one or more non-ionic surfactants selected from the group consisting of dimethylsulfoxide, ethanol, isopropanol, propylene glycol, glycerol monolaurate, propylene glycol monolaurate, sorbitan monolaurate, sorbitan sesquioleate, polysorbate 20, peg-40 stearate, steareth-20, poloxamer 185.

8. The intra-dermal formulation according to claim 1, where the formulation is a solution, cream, gel, ointment, lotion, spray, aerosol, foam or paste.

9. The intra-dermal formulation according to claim 1, wherein said anti-solvent is water.

10. The intra-dermal formulation according to claim 1 that is delivered to the skin by the use of a vehicle or device suitable for dermal or epidermal delivery.

11. The intra-dermal formulation according to claim 1 in the form of a solution, cream, ointment, lotion, gel, spray, aerosol, foam, or paste.

12. A method of inducing relief of neuropathic pain in a patient in need thereof, comprising applying to the skin of said patient a therapeutically effective amount of the compound 2-[2-(N-phenyl-N-2-indanyl)aminoethyl]piperidine, or an optically active isomer thereof or a pharmaceutically acceptable salt thereof, dissolved in one or more solvents in which said compound, isomer or salt is soluble, together with an anti-solvent in an amount sufficient to reduce the solubility of said compound, isomer or salt in said one or more solvents, wherein upon application of said formulation to the skin of said patient, said solvent evaporates to form a saturated solution of said compound, isomer or salt, causing said compound, isomer or salt to penetrate the stratum corneum and to accumulate in the epidermis and dermis of the skin in an amount effective for inducing relief of neuropathic pain.

13. A method of down-regulating the up-regulated sodium channels in thin dermal afferent nerve fibers and in dermal nerve endings of the skin of a patient suffering from neuropathic pain, comprising applying to the skin of said patient a therapeutically effective amount of the compound, or an optically active isomer thereof or pharmaceutically acceptable salt thereof, dissolved in one or more solvents in which said compound, isomer or salt is soluble, together with an anti-solvent in an amount sufficient to reduce the solubility of said compound, isomer or salt in said one or more solvents at least one of which is a volatile solvent, wherein upon application of said formulation to the skin of said patient, said volatile solvent evaporates to form a saturated solution of said compound, isomer or salt, causing said compound, isomer or salt to accumulate in the epidermis and dermis of the skin.
